(19) United States
(12) Patent Application Publication
(10) Pub. No.: US 2018/0117302 A1
(43) Pub. Date: May 3, 2018

(54) A WET/DRY CONVERTIBLE ELECTRODE AND METHOD OF USE

(71) Applicant: KONINKLIJKE PHILIPS N.V., EINDHOVEN (NL)

(72) Inventor: William Edwin CLEGG, Gibsonia, PA (US)

(21) Appl. No.: 15/571,935

(22) PCT Filed: Apr. 21, 2016
(86) PCT No.: PCT/IB2016/052256

§ 371 Date: Nov. 6, 2017

Related U.S. Application Data
(60) Provisional application No. 62/158,579, filed on May 8, 2015.

(51) Int. Cl. A61N 1/04; A61B 5/00; A61B 5/0408; A61B 5/0478; A61B 5/0492; A61B 5/0496

(52) U.S. Cl. CPC A61N 1/0496; A61B 5/6804; A61B 5/04087; A61B 5/0478; A61B 2562/125; A61B 5/0496; A61N 1/0452; A61N 1/0456; A61B 2562/0217; A61B 5/0492

(57) ABSTRACT

The present disclosure pertains to an electrode configured to provide electrical contact with skin of a subject. The electrode is a convertible electrode configured to function as a dry electrode and/or as a wet electrode according to the desired configuration. In a dry electrode configuration, the electrode includes a conductive portion, a receiving portion, and an electrical coupling. In a wet electrode configuration, the electrode includes conductive portion, receiving portion, a hydrogel material, and electrical coupling. The conductive portion is configured to receive and/or transmit electrical signals from and/or to the skin of the subject. The receiving portion is configured to receive the detachable hydrogel material. Without the hydrogel material, the electrode functions as a dry electrode. Responsive to the receiving portion receiving the detachable hydrogel material, the electrode functions as a wet electrode.

### BACKGROUND

### 1. Field

The present disclosure pertains to an electrode configured to provide electrical contact with skin of a subject.

### 2. Description of the Related Art

Electrodes that provide electrical contact with a subject's skin to transfer electrical signals between the subject's skin and a medical device are known. Typically electrodes are either dry electrodes or wet electrodes and are not convertible. Wet electrodes rely on an electrolyte material, such as a hydrogel or gel, to provide a conductive path and fill in gaps between the electrode and the skin of the subject. Dry electrodes rely on the natural salt and/or sweat on the skin of the subject to provide a flow path for the electrical signals transmitted to and/or received from the skin of the subject.

### SUMMARY

Accordingly, one or more aspects of the present disclosure relate to an electrode configured to provide electrical contact with skin of a subject. In one embodiment, the electrode comprises a conductive portion, a receiving portion, and an electrode coupling. The conductive portion is configured to receive electrical signals from and/or transmit electrical signals to the skin of the subject. The receiving portion is configured to receive a detachable hydrogel material. Without the hydrogel material the electrode functions as a dry electrode. Responsive to the receiving portion receiving the detachable hydrogel material, the electrode functions as a wet electrode. The electrical coupling facilitates coupling of the electrode to an external computing system.

Yet another aspect of the present disclosure relates to a method for providing electrical contact with skin of a subject via an electrode. The method comprises removably coupling a conductive portion of an electrode to the skin of a subject, facilitating coupling the electrode to an external computing system, and receiving and/or transmitting electrical signals. The conductive portion of the electrode is configured to receive electrical signals from and/or transmit electrical signals to the skin of the subject. The electrode includes a receiving portion configured to receive a detachable hydrogel material such that, without the hydrogel material the electrode functions as a dry electrode. Responsive to the receiving portion receiving the detachable hydrogel material, the electrode functions as a wet electrode.

Still another aspect of present disclosure relates to an electrode configured to provide electrical contact with skin of a subject. The electrode comprises means for transmitting and/or receiving electrical signals, means for receiving a detachable hydrogel material, and means for facilitating coupling of the electrode to an external computing system. The means for transmitting and/or receiving electrical signals include means for transmitting electrical signals to and/or receiving electrical signals from the skin of the subject. The means for receiving a detachable hydrogel material are configured such that, without the hydrogel material the electrode functions as a dry electrode. Responsive to the receipt of the detachable hydrogel material by the means for receiving, the electrode functions as a wet electrode.

These and other objects, features, and characteristics of the present disclosure, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the disclosure.

### BRIEF DESCRIPTION OF THE DRAWINGS

FIG. 1 illustrates an electrode in accordance with an embodiment;

FIG. 2 illustrates an electrode in accordance with another embodiment;

FIG. 3A illustrates a configuration of a hydrogel material in accordance with an embodiment;

FIG. 3B illustrates another configuration of a hydrogel material in accordance with an embodiment;

FIG. 3C illustrates another configuration of a hydrogel material in accordance with an embodiment;

FIG. 3D illustrates another configuration of a hydrogel material in accordance with an embodiment;

FIG. 4 illustrates an electrode in accordance with another embodiment; and

FIG. 5A illustrates a conductive portion in accordance with an embodiment;

FIG. 5B illustrates a cross section view of the conductive portion illustrated in FIG. 5A;

FIG. 6 illustrates a method for providing electrical contact with skin of a subject via an electrode, in accordance with an embodiment.

### DETAILED DESCRIPTION OF EXEMPLARY EMBODIMENTS

As used herein, the singular form of “a”, “an”, and “the” include plural references unless the context clearly dictates otherwise. As used herein, the statement that two or more parts or components are “coupled” shall mean that the parts are joined or operate together either directly or indirectly, i.e., through one or more intermediate parts or components, so long as a link occurs. As used herein, “directly coupled” means that two elements are directly in contact with each other. As used herein, “fixedly coupled” or “fixed” means that two components are coupled so as to move as one while maintaining a constant orientation relative to each other.

As used herein, the word “unitary” means a component is created as a single piece or unit. That is, a component that includes pieces that are created separately and then coupled together as a unit is not a “unitary” component or body. As employed herein, the statement that two or more parts or components “engage” one another shall mean that the parts exert a force against one another either directly or through one or more intermediate parts or components. As employed herein, the term “number” shall mean one or an integer greater than one (i.e., a plurality).

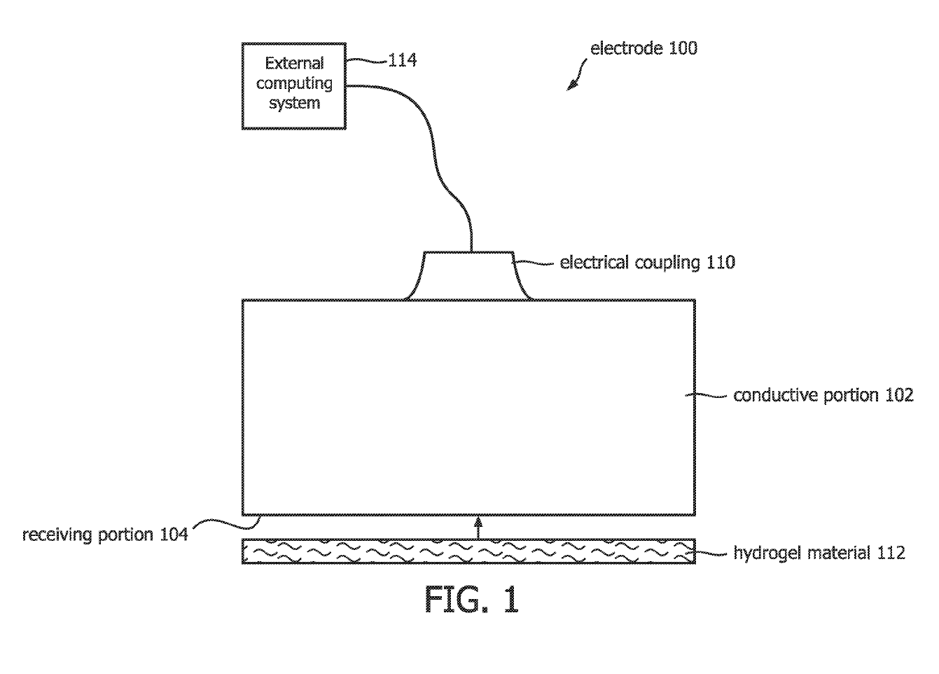

FIG. 1 illustrates an electrode 100 configured to provide electrical contact with skin of a subject. Electrode 100 provides an electrode that can be used as a dry electrode (e.g., without a hydrogel material) and/or a wet electrode (e.g., with a hydrogel material). Wet electrodes are typically used for shorter periods of time due to the hydrogel or gel drying out. Wet electrodes can cause irritation to the skin due to the gel material. Dry electrodes can be used for longer periods of time because they do not dry out, but they often have utility issues. For example, dry electrodes may be difficult to couple to the skin of the subject without gaps and may not stay in place well if a subject moves. A dry electrode may be more beneficial for some subjects based on their skin type, while others may do better with a wet electrode. Current electrodes are configured as either dry or wet and cannot accommodate for various applications and/or skin types. A wet/dry convertible electrode overcomes these deficiencies.

Electrode 100 is a convertible electrode configured to function as a dry electrode and/or as a wet electrode according to the desired configuration. In a dry electrode configuration, electrode 100 includes a conductive portion 102, a receiving portion 104, and an electrical coupling 110. In a wet electrode configuration, electrode 100 includes conductive portion 102, receiving portion 104, a hydrogel material 112, and electrical coupling 110. Conductive portion 110 is configured to receive and/or transmit electrical signals from and/or to the skin of the subject. Receiving portion 104 is configured to receive the detachable hydrogel material 112. Without hydrogel material 112, electrode 100 functions as a dry electrode. Responsive to receiving portion 104 receiving the detachable hydrogel material 112, electrode 100 functions as a wet electrode. Electrode 100 includes an electrical coupling that facilitates coupling the electrode to an external computing system 114. In some embodiments, hydrogel material 112 includes a stamped and/or cut hydrogel material. Hydrogel material 112 may be user configurable to include two or more layers and/or be based on the skin type of the subject. In some embodiments, electrode 100 is a fabric electrode with a fabric portion that includes conductive portion 102 and receiving portion 104.

As a convertible electrode able to function as a wet and/or dry electrode, electrode 100 can accommodate for various applications and/or subject skin types. A user (e.g., health care assistant or provider) can use the same electrode for subjects with sensitive skin that is irritated by hydrogel materials and for subjects with dry skin that need a hydrogel material. Also, electrode 100 may be converted from a dry electrode to a wet electrode and/or from a wet electrode to a dry electrode any number of times and at any point such that, if a treatment or procedure is not working as well as possible, the user can convert electrode 100 to improve the electrical contact with the skin of the subject. Further, the user configurability of electrode 100 enables hydrogel material 112 to be customizable based on the skin type of the subject. Thus, whether used as a dry electrode or a wet electrode, electrode 100 increases the applicability and customization of an electrode without sacrificing the utility.

As shown in FIG. 1, electrode 100 includes a conductive portion 102. Conductive portion 102 is configured to receive electrical signals from and/or transmit electrical signals to the skin of the subject. Conductive portion 102 removably couples to the skin of the subject to establish an electrical connection between the subject's skin and an external computing system 114 for delivering electrical stimulation to the subject, monitoring the physiological parameter of the subject, or both. Conductive portion 102 is removably coupled to the subject's skin via one or more of an adhesive, gel, tape, or other material. In some embodiments, conductive portion 102 removably couples to the skin of the subject as a portion of a larger structure such as a sleeve, garment, headband, hat, rigid apparatus, and/or other structure.

Conductive portion 102 may be a current spreader configured to spread current over the electrode. When coupled to the skin of the subject, a surface of conductive portion 102 faces the skin of the subject. Electrical signals are received from and/or transmitted to the skin of the subject via the surface of conductive portion 102 and/or conductive portion 102. In some embodiments, where electrode 100 is a fabric electrode, conductive portion 102 includes one or more of a fabric portion, a conductive surface, a metal coating, a transfer layer, and/or other components described herein according to one or more embodiments.

Receiving portion 104 is configured to receive a detachable hydrogel material 112. Without hydrogel material 112, electrode 100 functions as a dry electrode. Without hydrogel material 112, natural salt and/or sweat on the skin of the subject provide a flow path for the electrical signals transmitted to and/or received from the skin of the subject. Responsive to receiving portion 104 receiving detachable hydrogel material 112, electrode 100 functions as a wet electrode. Hydrogel material 112 provides a conductive flow path for the electrical signals received from and/or transmitted to the skin of the subject. Thus, electrode 100 is a convertible electrode.

In some embodiments, receiving portion 104 is the surface of conductive portion 102 that faces the skin of the subject responsive to electrode 100 being coupled to the subject. In other embodiments, receiving portion 104 includes a cavity disposed between the skin of the subject and conductive portion 102 responsive to electrode 100 being coupled to the skin of the subject. The cavity is comprised of one or both of conductive portion 102 and/or other portions of electrode 100. For example, the cavity may be formed by a ridge around the perimeter of the surface of conductive portion 102. The ridge may be part of conductive portion 102 or separately coupled with conductive portion 102. In some embodiments, the cavity comprises one or more geometric shapes and/or one or more depths.

Configured as a wet electrode, electrode 100 includes a hydrogel material 112. Hydrogel material 112 includes a hydrogel and/or electrolyte material that provides a conductive flow path for the transfer of electrical signals. The hydrogel and/or electrolyte material comprise one or more of glycerin, water, polyacrylate, and/or other ingredients. Hydrogel material 112 provides a conductive flow path for the electrical signals received from and/or transmitted to the skin of the subject. Responsive to electrode 100 being removably coupled to the subject, hydrogel material 112 is disposed on receiving portion 104 and the skin of the subject. In some embodiments, hydrogel material 112 acts as an adhesive configured to couple electrode 100, conductive portion 102, and/or receiving portion 104 to the skin of the subject. In other embodiments, hydrogel material 112 may include an adhesive distinct from the hydrogel and/or electrolyte material.

In some embodiments, hydrogel material 112 is pre-stamped and/or pre-cut such that it is configured to fit into, match up with, cover, and/or otherwise couple with receiving portion 104. In other embodiments, hydrogel material 112 includes a sheet of hydrogel material that a user or operator has to cut and/or stamp into the desired shape. Hydrogel material 112 is coupled with receiving portion 104 by the user and/or operator. In one embodiment, the user and/or operator couples the stamped and/or cut portion of hydrogel material 112 to receiving portion 104 by placing it against receiving portion 504.

Hydrogel material 112 may include one or more hydrogel materials arranged in one or more layers. For example, a hydrogel material may include a saline solution. In some embodiments, various hydrogel materials may include various properties. Hydrogel material 112 may be user configurable such that a user can select one or more hydrogel materials and/or one or more layers of the hydrogel materials to couple with electrode 100. In some embodiments, the user and/or operator may customize one or more layers of hydrogel material 112. For example, a user can couple one or more layers of hydrogel material 112 to receiving portion 104 as desired. The one or more layers of hydrogel material 112 may be determined based on the skin type of the subject. As such, a user may choose a hydrophilic or a hydrophobic hydrogel material 112 to couple to receiving portion 104 based on the skin type of the subject. By way of illustration, a hydrogel used for a subject with oily skin may differ from a hydrogel used for a subject with dry skin. A hydrogel may have a variety of conductivity levels and/or adhesive properties.

Configured as a dry electrode, electrode 100 does not include hydrogel material 112. Without hydrogel material 112, electrode 100 operates as a dry electrode and may use the sweat and/or salt on the skin of a subject to provide a conductive flow path for the electrical signals received from and/or transmitted to the skin of the subject. Whether or not electrode 100 includes hydrogel material 112 may be determined by the user such that the user may convert electrode 100 from a dry electrode (e.g., without hydrogel material 112) to a wet electrode (e.g., with hydrogel material 112).

Electrode 100 includes electrical coupling 110. Electrical coupling 110 facilitates coupling of electrode 100 to an external computing system 114. External computing system 114 is a system configured to deliver electrical stimulation to a subject and/or monitor a physiological parameter or such of the subject. In some embodiments, external computing system includes one or more of processing devices (e.g., a digital processor, an analog processor, a digital circuit designed to process information, an analog circuit designed to process information, a state machine, and/or other mechanisms for electronically processing information), one or more sensors, one or more interface devices (e.g., a keypad, buttons, switches, a keyboard, knobs, levers, a display screen, a touch screen, speakers, a microphone, an indicator light, an audible alarm, a printer, a tactile feedback device, and/or other interface devices), and/or other components.

For example, external system 114 may include a bio sensing systems used for electrocardiography (ECG), electroencephalography (EEG), electromyography (EMG), electrooculography (EOG), and/or other bio sensing applications. By way of another example, external system 114 may include a bio stimulation system used for transcutaneous electrical nerve stimulation (TENS), electrical muscle stimulation (EMS), neuromuscular electrical stimulation (NMES), functional electrical stimulation (FES), and/or other bio stimulation applications.

Electrical coupling 110 is electrically coupled to conductive portion 102 and/or receiving portion 104 such that it enables electrical signals to be received and/or transmitted from and/or to the skin of the subject. In some embodiments, additional components and/or layers of material are disposed between electrical coupling 110, conductive portion 102, and/or receiving portion 104. In other embodiments, electrical coupling 110 is directly coupled to conductive portion 102. Electrical coupling 110 comprises one or more of a snap assembly, a magnetic assembly, a button assembly, a clip and/or clamp assembly, a wire assembly, and/or other assemblies to facilitate coupling electrode 100 to external computing system 114. For example, electrical coupling 110 includes a portion of a snap assembly for connecting one or more wires from external computing system 114 to electrode 100.

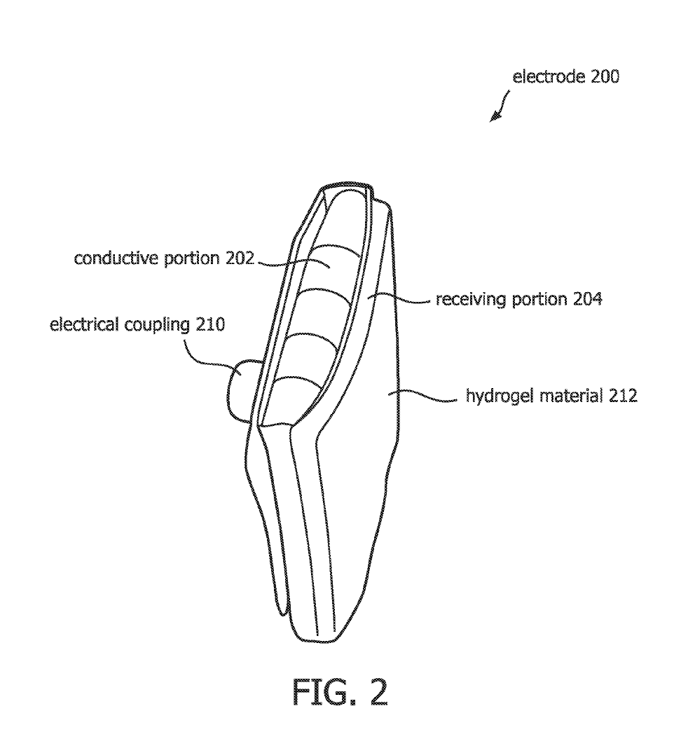

FIG. 2 illustrates an electrode 200 configured to provide electrical contact with skin of a subject according to an embodiment. Electrode 200 includes a conductive portion 202, a receiving portion 204, an electrical coupling 210, a hydrogel 212, and/or other components. In some embodiments, electrode 200 may include only a portion of the components discussed herein and/or additional components not discussed herein.

In some embodiments, conductive portion 202 comprises a fabric. In some embodiments, the fabric may include a surface modified fabric. The fabric may be folded to include multiple layers of the fabric. In some embodiments, conductive portion 202 includes the fabric that is folded around one or more alternate material layers (e.g., a foam layer) that do not interfere with the transmission and/or receipt of electrical signals to and/or from the skin of the subject. Conductive portion 202 is configured to receive electrical signals from and/or transmit electrical signals to the skin of the subject. Conductive portion 202 includes a surface configured to face toward the skin of the subject such that the electrical signals are received and/or transmitted via the surface of conductive portion 202.

According to the illustrated embodiment, receiving portion 204 includes and/or is the same as the surface of conductive portion 202 that is configured to face toward the skin of the subject. In other embodiments, receiving portion 204 may not be the same as the surface of conductive portion 202 configured to face toward the skin of the subject. For example, receiving portion may be a cavity. Receiving portion 204 is configured to receive detachable hydrogel material 212 by providing a surface to couple to hydrogel material 212.

As discussed herein, hydrogel material 212 provides a conductive flow path for the electrical signals received from and/or transmitted to the skin of the subject. Hydrogel material 212 is pre-cut and/or pre-stamped according to receiving portion 204. Hydrogel material 212 may act as an adhesive and couple itself to receiving portion 204. Where hydrogel material 212 does not have adhesive properties, other adhesives and/or structures may facilitate coupling of hydrogel material 212 to receiving portion 204. As illustrated in FIG. 2, hydrogel material 212 is configured such covers receiving portion 204 when coupled to it.

Electrode 200 includes electrical coupling 210 that facilitates coupling of electrode 200 to an external computing system via one or more corresponding wire couplings. As illustrated in the embodiment of FIG. 2, electrical coupling 210 may be a male portion of a snap and a corresponding female portion of a snap may be coupled to one or more wires configured to couple electrode 200 to the external computing system.

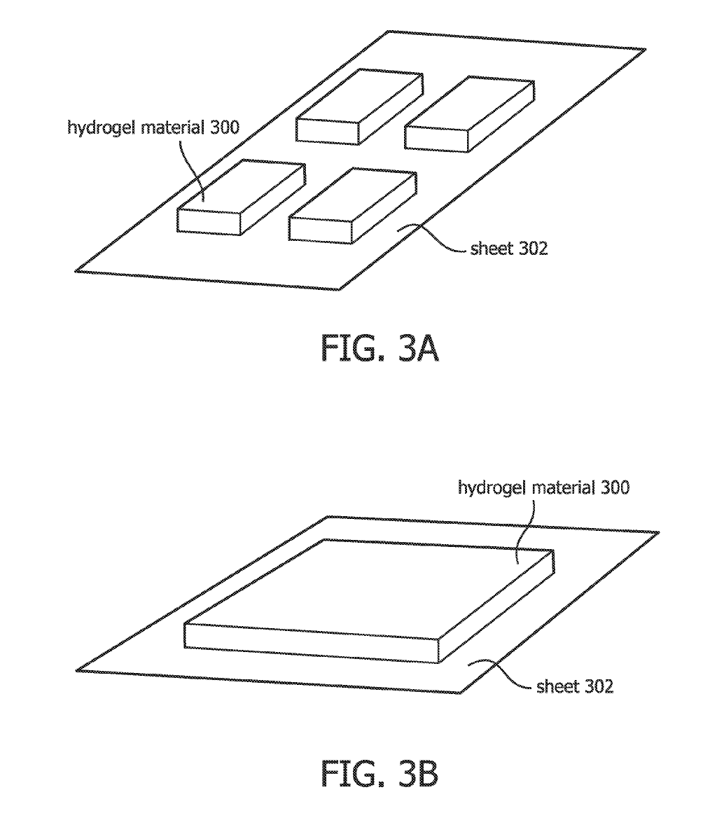

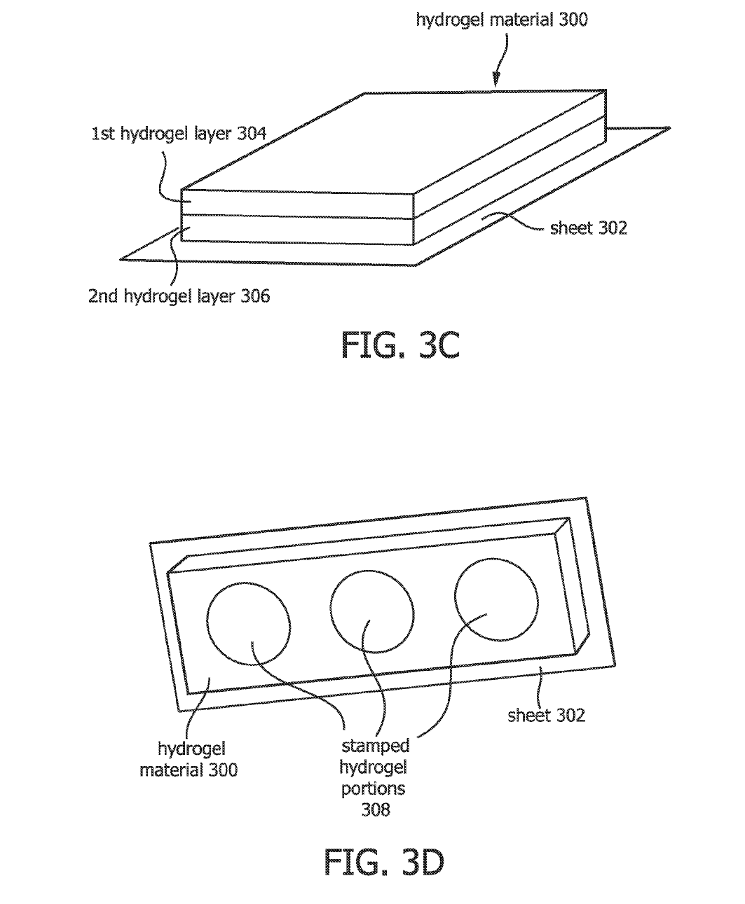

FIG. 3A illustrates a configuration of a hydrogel material 300 in accordance with an embodiment. Hydrogel material 300 is cut such that it may be received by a receiving portion of an electrode. The cut portions of hydrogel 300 have a shape corresponding to the receiving portion of the electrode. For example, hydrogel 300 is cut as rectangular portions for a rectangular shaped receiving portion. Hydrogel material 300 is on sheet 302 and is configured to be removed from sheet 302 by a user. Sheet 302 may be the same size as, smaller than, or larger than hydrogel material 300. In some embodiments, each cut portion of hydrogel material 300 may be on a separate sheet. An additional sheet the same as or similar to sheet 302 may cover the top surface of hydrogel material 300.

FIG. 3B illustrates a configuration of a hydrogel material 300 in accordance with an embodiment. Hydrogel material 300 is a sheet of hydrogel material such that a user may cut it as desired. The user may cut portions of hydrogel 300 according to a receiving portion of an electrode. Hydrogel material 300 is on sheet 302 and is configured to be removed from sheet 302 by a user. Sheet 302 may be the same size as, smaller than, or larger than hydrogel material 300. In some embodiments, an additional sheet the same as or similar to sheet 302 may cover the top surface of hydrogel material 300.

FIG. 3C illustrates a configuration of a hydrogel material 300 in accordance with an embodiment. Hydrogel material 300 includes a first hydrogel layer 304 and a second hydrogel layer 306. First hydrogel layer 304 and a second hydrogel layer 306 may comprise different hydrogels, ingredients, compositions, and/or electrolyte materials. First hydrogel layer 304 may have different properties than second hydrogel layer 306. Hydrogel 300 may be cut such that it may be received by a receiving portion of an electrode. In some embodiments, one or both of first hydrogel layer 304 and/or second hydrogel layer 306 is/are configured to detachably couple to the receiving portion of an electrode such that hydrogel material 300 is reversible. Hydrogel material 300 is on sheet 302 and is configured to be removed from sheet 302 by a user. Sheet 302 may be the same size as, smaller than, or larger than hydrogel material 300. In some embodiments, an additional sheet the same as or similar to sheet 302 may cover the top surface of hydrogel material 300.

FIG. 3D illustrates a configuration of a hydrogel material 300 in accordance with an embodiment. Hydrogel material 300 includes stamped hydrogel portions 308. Hydrogel material 300 is stamped such that it may be received by a receiving portion of an electrode. The stamped hydrogel portions 308 have a shape corresponding to the receiving portion of the electrode. For example, stamped portions 308 are stamped into circular shapes for a circular shaped receiving portion. Hydrogel material 300 is on sheet 302 and is configured to be removed from sheet 302 by a user. Sheet 302 may be the same size as, smaller than, or larger than hydrogel material 300. In some embodiments, an additional sheet the same as or similar to sheet 302 may cover the top surface of hydrogel material 300.

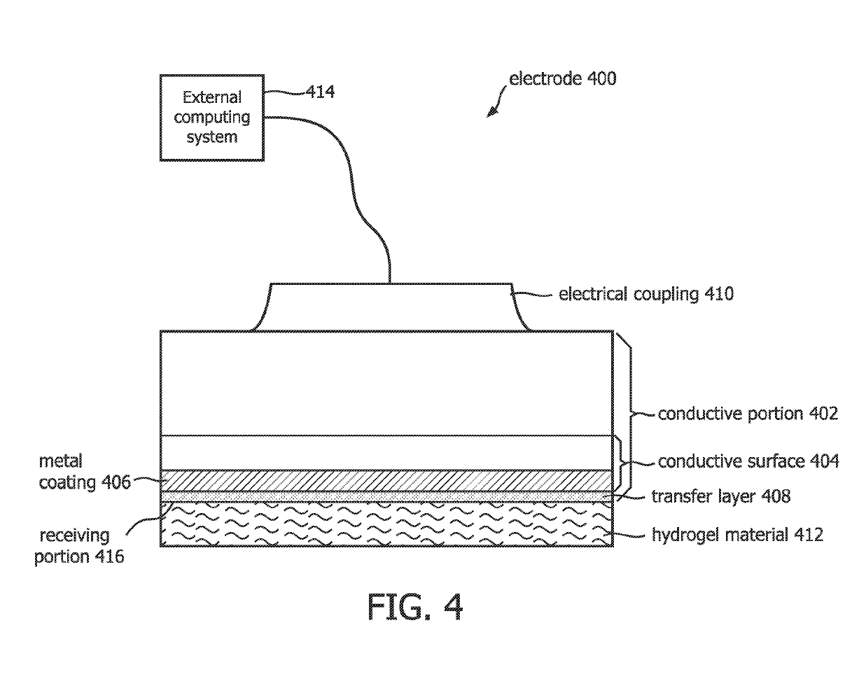

FIG. 4 illustrates an electrode in accordance with another embodiment. Electrode 400 includes a conductive portion 402, a receiving portion 416, electrical coupling 410, and/or other components. In the illustrated embodiment, conductive portion 402 is comprised of fabric. In some embodiments, the fabric includes a surface modified fabric. Conductive portion 402 includes one or more of a conductive surface 404, a metal coating 406, a transfer layer 408, and/or other components.

Conductive portion 402 includes a woven fabric including one or more materials. Conductive portion 402 partially comprises one or more of cotton, polyester, nylon, and/or other materials. In some embodiments, conductive portion 402 is cut, torn and/or folded. Conductive portion 402 is also glued, taped, layered, torn, tied, melted, pinned, sewn, and/or otherwise configured to couple with another portion of electrode 400 and/or to itself. Conductive portion 402 is configured to removably couple to the skin of the subject. In some embodiments, conductive portion is flexible and conforms to the shape of the subject. Conductive portion 402 removably couples to the skin of the subject to establish an electrical connection between the subject's skin, the electrode, and/or an external computing system 414 for delivering electrical stimulation to the subject, monitoring the physiological parameter of the subject, or both. Conductive portion 402 is removably coupled to the subject's skin via one or more of an adhesive, gel, tape, or other material. In some embodiments, conductive portion 402 removably couples to the skin of the subject as a portion of a larger structure such as a sleeve, garment, headband, hat, and/or other structure.

Conductive portion 402 includes conductive surface 404. Conductive surface 404 receives electrical signals from and/or transmits electrical signals to the skin of the subject. Responsive to the electrode being coupled with the skin of the subject, conductive surface 404 faces toward the skin of the subject. Conductive surface 404 may be a current spreader configured to spread current over the electrode. Conductive surface 404 comprises at least a portion of a surface of conductive portion 402. In some embodiments, conductive surface 404 covers all of a surface of conductive portion 402.

Conductive surface 404 includes a metal coating 406. In some embodiments, conductive portion 402 and conductive surface 404 include metal coating 406. Metal coating 406 is comprised of a layer of one or more metals. For example, metal coating 406 may include a layer of silver, tin, gold, platinum, stainless steel, or other metals. In some embodiments, metal coating 406 may not comprise metal but comprises other materials than can be coated onto a fabric. Metal coating 406 covers at least a portion of conductive surface 404 and conductive portion 402. Responsive to electrode 400 being coupled to the subject, metal coating 406 faces toward the skin of the subject.

In some embodiments, metal coating 406 is on one side of, both sides of, and/or throughout conductive portion 402. As such, conductive portion 402 and metal coating 406 comprise a metal coated (e.g., metallized) fabric. In an example embodiment, the metallized fabric of conductive portion 402 includes one or more of the following properties: a surface resistance of less than 1 Ohms, an ion release rate between 80-90%, a bacterial kill rate of 99% in the first ten minutes, and/or other properties.

Metal coating 406 includes a transfer layer 408. Transfer layer 408 includes a portion of metal coating 406 that is configured to facilitate ion transfer between electrode 400 and the skin of the subject. In some embodiments, transfer layer 404 is a thin coating on metal coating 406. In some embodiments, transfer layer 404 may include a portion of metal coating 406. Transfer layer 404 includes properties that enable ion transfer and use as an electrode. For example, when metal coating 406 includes a silver coating, transfer layer 408 may include silver chloride. Imparting a silver chloride structure to a portion of conductive surface 404 as part of conductive portion 402 enables the use of fabric as an electrode.

In some embodiments, a thickness of transfer layer 408 may be smaller than a thickness of metal coating 406. As such, transfer layer 408 includes a thin portion of the surface of metal coating 406. In some embodiments, transfer layer 408 does not have a uniform thickness and/or transfer layer 408 only partially covers the surface of metal coating 406. In other embodiments, transfer layer 408 includes a thicker portion of metal coating 406. Where metal coating 406 is on both sides of and/or throughout conductive portion 402, transfer layer 408 is on both sides of and/or throughout conductive portion 402.

Receiving portion 416 is configured to receive a detachable hydrogel material 412. Without hydrogel material 412, electrode 400 functions as a dry electrode. In some embodiments, conductive surface 404 (including metal coating 406 and transfer layer 408) is receiving portion 416. Receiving portion 416 is configured to receive detachable hydrogel material 412 by providing a surface (e.g., conductive surface 404) to couple to hydrogel material 412. In other embodiments, receiving portion 416 includes a cavity disposed between the skin of the subject and conductive surface 404 and/or conductive portion 402 responsive to electrode 400 being coupled to the skin of the subject.

Although illustrated in FIG. 4 in the form of layers, conductive portion 402, conductive surface 404, metal coating 406, transfer layer 408, and/or hydrogel material 412 are not intended to be limited to a layered configuration as depicted. For example, metal coating 406 and transfer layer 408 may cover multiple surfaces of conductive portion 402.

Electrode 400 includes electrical coupling 410. Electrical coupling 410 facilitates coupling of electrode 400 to an external computing system 414. As discussed herein, external computing system 414 is a system configured to deliver electrical stimulation to a subject and/or monitor a physiological parameter or such of the subject. Electrical coupling 410 is electrically coupled to conductive portion 402 and/or conductive surface 404 such that it enables electrical signals to be received and/or transmitted from and/or to the skin of the subject. In some embodiments, additional components and/or layers of material are disposed between electrical coupling 410, conductive portion 402, and/or conductive surface 404. In other embodiments, electrical coupling 410 is directly coupled to conductive portion 402 and/or conductive surface 404. Electrical coupling 410 comprises one or more of a snap assembly, a magnetic assembly, a button assembly, a clip and/or clamp assembly, a wire assembly, and/or other assemblies to facilitate coupling electrode 400 to external computing system 414. For example, electrical coupling 410 includes a portion of a snap assembly for connecting one or more wires from external computing system 414 to electrode 400.

Configured as a wet electrode, electrode 400 includes hydrogel material 412. Hydrogel material 412 provides a conductive flow path for the electrical signals received from and/or transmitted to the skin of the subject. Configured as a dry electrode, electrode 400 does not include hydrogel material 412. Whether or not electrode 400 includes hydrogel material 412 may be determined by the user such that the user may convert electrode 400 from a dry electrode (e.g., without hydrogel material 412) to a wet electrode (e.g., with hydrogel material 412).

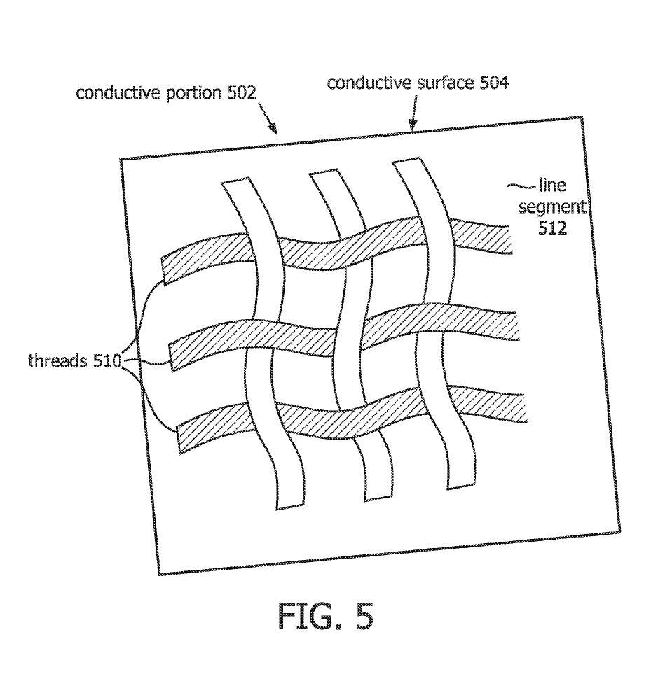

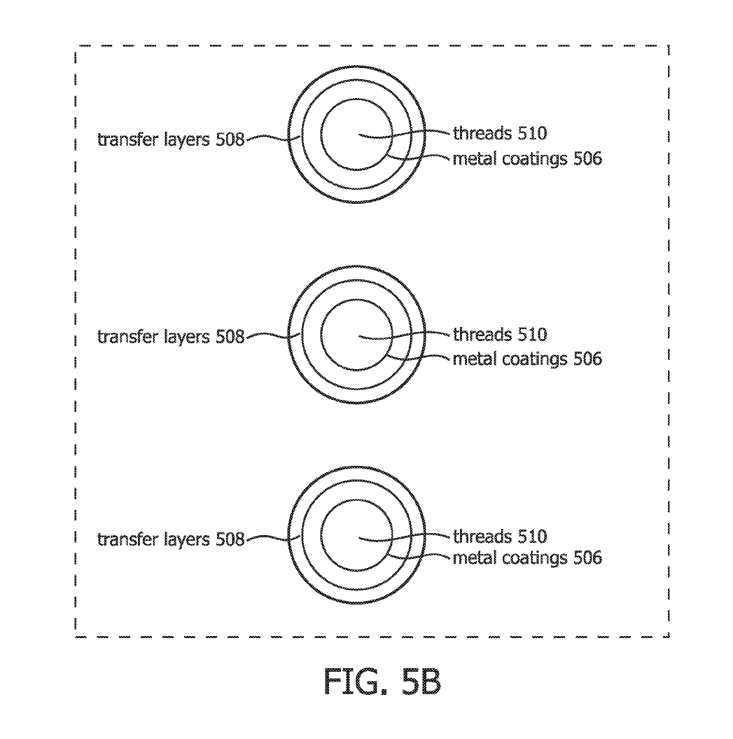

FIG. 5A illustrates a conductive portion 502 comprised of fabric. Conductive portion 502 includes a conductive surface 504 configured to provide electrical contact with skin of a subject according to an embodiment. According to the illustrated embodiment, conductive surface 504 comprises one or more threads 510 having metal coatings 506 and transfer layers 508. Conductive portion 502 includes metal coatings 506 that are applied to the surface of one or more threads 510. One or more threads 510 including metal coatings 506 comprise at least a portion of the fabric comprising conductive portion 502. In some embodiments, one or more threads 510 including metal coatings 506 are woven together with complimentary materials to form conductive portion 502. As such, metal coatings 506 are on both sides of and throughout conductive portion 502.

In the embodiment illustrated in FIG. 5A, metal coatings 506 that cover one or more threads 510 may include transfer layers 508. Transfer layers 508 include a portion of metal coatings 506 that are configured to facilitate ion transfer between electrode 500 and the skin of the subject. In some embodiments transfer layers 508 include properties that facilitate use as an electrode. For example, when metal coatings 506 include silver coatings, transfer layers 508 may include silver chloride. In some embodiments, a silver chloride structure, may be imparted to the surface of the silver coatings to create transfer layers 508. Imparting a silver chloride structure to a portion of conductive surface 504 as part of conductive portion 502 enables the use of fabric as an electrode. In some embodiments, transfer layers 508 include a thin portion of the circumferential surface of metal coatings 506.

A cross section of conductive portion 502 at line segment 512 is illustrated in FIG. 5B. The cross section view of one or more threads 510 illustrates metal coatings 506 covering the circumferential surface area of one or more threads 510. Transfer layers 508 include at least portions of the circumferential surface area of metal coatings 506. One or more threads 510 are woven with other materials to create the fabric of conductive portion 502 may include transfer layers 508.

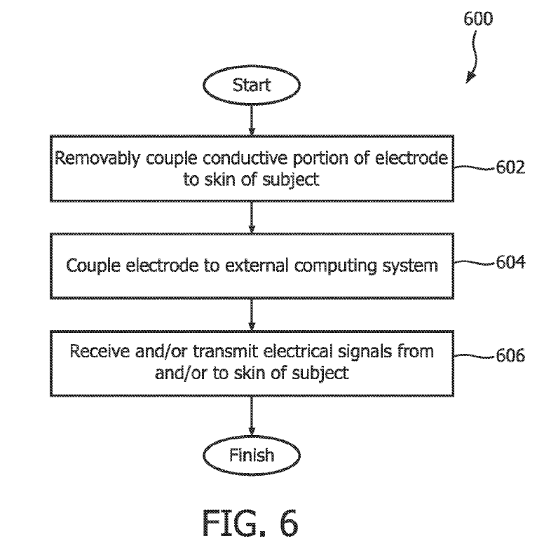

FIG. 6 illustrates a method 600 for providing electrical contact with skin of a subject via an electrode. The electrode comprises one or more of a conductive portion, a receiving portion, an electrical coupling, and/or other components. In some embodiments, more than one electrode is used. The operations of method 600 presented herein are intended to be illustrative. In some embodiments, method 600 may be accomplished with one or more additional operations not described, and/or without one or more of the operations discussed. Additionally, the order in which the operations of method 600 are illustrated in FIG. 6 and described herein is not intended to be limiting.

At an operation 602, the conductive portion of the electrode is coupled with the skin of the subject. Operation 602 is performed by a conductive portion the same as or similar to conductive portion 102 (shown in FIG. 1 and described herein).

At an operation 604, coupling of the electrode is coupled to an external computing system. In some embodiments, coupling of the electrode to an external computing system is facilitated via an electrical coupling the same as or similar to electrical coupling 110 (shown in FIG. 1 and described herein).

At operation 606, electrical signals are received and/or transmitted from and/or to the skin of the subject via the conductive portion of the electrode. The electrode includes the receiving portion which is configured to receive a detachable hydrogel material. Without the hydrogel material, the electrode functions as a dry electrode. Responsive to the receiving portion receiving a detachable hydrogel material, the electrode functions as a wet electrode. In some embodiments, operation 606 is performed by a conductive portion the same as or similar to conductive portion 102 and an electrode the same as or similar to electrode 100 (shown in FIG. 1 and described herein).

In some embodiments, as part of operation 602, the stamped or cut hydrogel material is detachably coupled to the receiving portion of the electrode. In some embodiments, this portion of operation 602 is performed by a hydrogel material the same as or similar to hydrogel material 112 (shown in FIG. 1 and described herein).

Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

In the claims, any reference signs placed between parentheses shall not be construed as limiting the claim. The word “comprising” or “including” does not exclude the presence of elements or steps other than those listed in a claim. In a device claim enumerating several means, several of these means may be embodied by one and the same item of hardware. The word “a” or “an” preceding an element does not exclude the presence of a plurality of such elements. In any device claim enumerating several means, several of these means may be embodied by one and the same item of hardware. The mere fact that certain elements are recited in mutually different dependent claims does not indicate that these elements cannot be used in combination.

Although the description provided herein provides detail for the purpose of illustration based on what is currently considered to be the most practical and preferred embodiments, it is to be understood that such detail is solely for that purpose and that the disclosure is not limited to the expressly disclosed embodiments, but, on the contrary, is intended to cover modifications and equivalent arrangements that are within the spirit and scope of the appended claims. For example, it is to be understood that the present disclosure contemplates that, to the extent possible, one or more features of any embodiment can be combined with one or more features of any other embodiment.

1. An electrode assembly, including an electrode and an attachable/detachable hydrogel material, configured to provide electrical contact with skin of a subject, the electrode assembly comprising:
an electrode having a conductive portion configured to receive electrical signals from and/or transmit electrical signals to the skin of the subject;
a stamped and/or cut hydrogel material configured to be removable from a sheet so as to be coupled to the electrode;
a receiving portion of the electrode configured to detachably receive the hydrogel material such that, without the hydrogel material the electrode functions as a dry electrode and, responsive to the receiving portion receiving the hydrogel material, the electrode functions as a wet electrode; and
an electrical coupling that facilitates coupling the electrode to an external computing system.

2. The electrode assembly of claim 1, wherein the receiving portion includes a cavity formed by a ridge around a perimeter of a conductive surface of the conductive portion.

3. The electrode assembly of claim 1, wherein the hydrogel material includes two or more layers, and wherein the two or more layers comprise different materials.

4. The electrode assembly of claim 1, wherein the electrode is a fabric electrode including a fabric portion that includes the conductive portion and the receiving portion, wherein each thread of the fabric portion contacting a conductive surface of the conductive portion is circumferentially coated with a metal, which in turn is circumferentially coated with a transfer layer.

5. The electrode assembly of claim 1, wherein the conductive portion comprises fabric.

6. A method for providing electrical contact with skin of a subject via an electrode, the electrode comprising a conductive portion, a receiving portion, and an electrical coupling, the method comprising:
removably coupling the conductive portion of the electrode to the skin of a subject;
coupling the electrode to an external computing system via the electrical coupling; and
receiving and/or transmitting electrical signals from and/or to the skin of the subject via the conductive portion of the electrode, the electrode including the receiving portion configured to receive a detachable hydrogel material including one or more layers such that, without the hydrogel material the electrode functions as a dry electrode and, responsive to the receiving portion receiving the detachable hydrogel material, the electrode functions as a wet electrode, wherein a) whether the hydrogel material is used or not is determined based on a skin type of the subject, and b) if the hydrogel material is used then a type of hydrogel and/or a number of layers of the hydrogel material are selected based on the skin type of the subject.

7. The method of claim 6, further comprising detachably coupling a stamped and/or cut hydrogel material to the receiving portion of the electrode.

8. The method of claim 6, wherein the hydrogel material includes two or more layers, and wherein the two or more layers comprise different materials.

9. The method of claim 6, wherein the electrode is a fabric electrode including a fabric portion that includes the conductive portion and the receiving portion, wherein each thread of the fabric portion contacting a conductive surface of the conductive portion is circumferentially coated with a metal, which in turn is circumferentially coated with a transfer layer.

10. The method of claim 6, wherein the conductive portion comprises fabric.

11. An electrode assembly, including an electrode and an attachable/detachable hydrogel material, configured to provide electrical contact with skin of a subject, the electrode assembly comprising:
an electrode having means for transmitting electrical signals to and/or receiving electrical signals from the skin of the subject;
means for providing a conductive flow path for the electrical signals received from and/or transmitted to the skin of the subject, the means for providing including a hydrogel material that is stamped and/or cut and configured to be removable from a sheet so as to be coupled to the electrode;
means of the electrode for detachably receiving the hydrogel material such that, without the hydrogel material the electrode functions as a dry electrode and, responsive to the receipt of the hydrogel material by the means for receiving, the electrode functions as a wet electrode; and
means for facilitating coupling of the electrode to an external computing system.

12. The electrode of claim 11, wherein the means for detachably receiving the hydrogel material includes a cavity formed by a ridge around a perimeter of a conductive surface of the means for transmitting.

13. The electrode of claim 11, wherein the means for providing includes two or more layers, and wherein the two or more layers comprise different materials.

14. The electrode of claim 11, wherein the electrode is a fabric electrode including a fabric portion that includes the means for transmitting and/or receiving and the means for receiving the hydrogel material, wherein each thread of the fabric portion contacting a conductive surface of the means for transmitting and/or receiving is circumferentially coated with a metal, which in turn is circumferentially coated with a transfer layer.

15. The electrode of claim 11, wherein the means for transmitting and/or receiving are at least partially comprised of fabric.
